(19) United States
(12) Patent Application Publication
(10) Pub. No.: US 2005/0273269 A1
(43) Pub. Date: Dec. 8, 2005

(54) Method and system for analysis of biological and chemical data

(76) Inventors: Zohar Yakhini, Ramat Hasharon (IL); Anya Tsalenko, Chicago, IL (US); Amir Ben-Dor, Bellevue, WA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/863,045

(22) Filed: Jun. 7, 2004

(51) Int. Cl. G06F 19/00; G01N 33/48; G01N 33/50

(52) U.S. Cl. 702/19; 702/27

(57) ABSTRACT

In various embodiments of the present invention, initial experimental data is initially partitioned into classes by sample source, concentration or number-of-molecule values are computed with respect to each initial partition, and a rank consistency score or fold-change consistency score is computed for various molecular concentration or number-of-copies determinants with respect to one or more class-specifying events of interest. In other words, rather than partitioning experimental data directly into two or more classes relative to an event of interest, the experimental data is first partitioned according to sample source, and then each sample-source partition is partitioned into two or more classes relative to an event of interest.

The present invention is related to analysis of experimental data and, in particular, to a method and system for using experimental data separately processed for each sample source in a multi-sample-source data set to facilitate identification of particular molecular-abundance determinants, including methods and system for using gene-expression data separately processed for each sample source in a gene-expression data set to facilitate identification of particular genes that exhibit significant differential expression in response to particular events, environmental changes, drug treatments, and other such phenomena.

### BACKGROUND OF THE INVENTION

During the past decade, phenomenal progress has been made in identifying and characterizing the genetic components of particular biological organisms, including humans, and in developing tools and methodologies for rapid analysis of gene-expression levels in biological tissue samples. One important, relatively recently developed tool for gene-expression-analysis is the microarray, a wafer-like substrate on which are arrayed thousands of features, each containing a particular type of probe molecule targeting a particular biopolymer sequence. Exposure of a microarray to a suitably prepared and labeled sample of copy deoxyribonucleic acid (“cDNA”) prepared from messenger ribonucleic acid (“mRNA”) isolated and purified from tissue samples allows for rapid determination of the expression levels of hundreds, thousands, or tens of thousands of different genes, depending on the size and contents of the microarray used. Repeated microarray-based experiments can be used to determine gene-expression levels of thousands or tens of thousands of genes within a biological tissue at discrete points in time. Determination of gene-expression levels at various time points over the course of a change in, or before and after a perturbation to, a biological organism or tissue allows for correlation of gene-expression levels with the change or perturbation. In particular, researchers, clinicians, and diagnosticians seek to identify particular genes that are differentially expressed with respect to a particular change or perturbation. For example, researchers and medical diagnosticians may seek to identify genes differentially expressed in nascent tumor tissue, in order to develop diagnostic tests to detect the onset of tumor growth. As another example, particular genes differentially expressed in response to exposure of biological tissues to a particular drug may allow clinicians to carefully monitor and determine the exposure levels to various different types of tissues and organs within a biological organism resulting from a particular drug-therapy regime. In view of the importance of gene-expression analysis, the present invention is discussed with respect to gene-expression analysis, although the present invention is far more widely applicable to analysis of factors responsible for observed concentrations or numbers of copies of various, particular biopolymers and molecules in sample solutions obtained by experimental means. For example, the present invention may be applied to proteomics experiments conducted using protein arrays, experimental analysis of polysaccharides, experimental analysis of other types of biopolymers, and experimental analysis of small-molecule components of biological and chemical systems. In many biological, experimental systems, genes may be considered to be ultimate molecular abundance determinants, although, in other experimental systems, other factors, including gene-expression regulators, catalytic proteins, conformation-altering proteins, and other entities may be considered to be molecular-abundance determinants.

FIG. 1 shows a matrix representation of a gene-expression data set. As shown in FIG. 1, the gene-expression data may be thought of as being tabulated in a two-dimensional matrix E 100. Each row in the matrix E, such as the first row 102, contains expression levels measured for a particular gene. In FIG. 1, the matrix E 100 includes N rows corresponding to N different genes, the expression levels for which have been determined in a number of different experiments. The matrix E 100 includes M different columns, such as column 104, each column containing the expression levels measured for up to N different genes in a single experiment, generally for a single sample. In fact, the gene-expression levels for multiple samples may be determined using a single microarray and different labels for each sample, and samples may be mixtures of biopolymers isolated from different tissues or organisms at different times. However, for simplicity of discussion and illustration, it is assumed, in the following discussion, that each column of matrix E represents a gene-expression-level determination for up to N genes at a discrete point in time for a particular sample. The different genes, as well as the rows in matrix E, are indexed by the index integer i, where the value of i ranges from between 1 and N. The notation Eᵢ refers to the gene-expression-level data for gene i. The different experiments or samples, as well as the columns in matrix E, are indexed by the indexing integer j, where the value of j ranges from between 1 and M A column may be alternatively referred to as “sample j” or as “experiment j.” As shown in FIG. 1, a particular cell E_{i,j} or E(i,j) 106 in the gene-expression-data matrix E corresponds to the expression level for the i^{th} gene measured in the j^{th} experiment or j^{th} sample.

Currently, in searching for genes differentially expressed with respect to a particular event, change, perturbation, drug exposure, environmental change, pathology, or other condition or phenomena, referred to below collectively as “event,” the gene-expression-data matrix E is partitioned into two or more submatrices, each corresponding to those experiments that measure gene-expression data for a particular event state. For example, the gene-expression-data matrix E may be partitioned into a submatrix B, or before class B, containing experimental data collected from tissues prior to exposure of the tissues to a particular drug, and a submatrix A, or after class A, containing experimental data collected from tissues following exposure of the tissues to a particular drug. FIG. 2 illustrates partitioning of a gene-expression data matrix E into two submatrices, or classes. In FIG. 2, an initial gene-expression-data matrix E 200 is partitioned into submatrices B 202 and A 204, where the submatrix B contains gene-expression data collected in M_{B} experiments before a particular event, and submatrix A 204 includes the gene-expression data collected in M_{A} experiments after a particular event. Although a two-class hypothetical partitioning relative to a single event is used in the following discussion, partitioning into three or more classes with respect to two or more events may be employed to seek genes that are differentially expressed relative to two or more events.

FIG. 3 illustrates the gene-expression data for a particular gene contained within the initial gene-expression-data matrix E and within submatrices B and A. In FIG. 3, the horizontal, double-headed arrows 302-304 pass through the centers of the rows of matrices E, B, and A corresponding to the 9^{th} gene, or the gene indexed by i=9. An important task for researchers, diagnosticians, and clinicians, as discussed above, is to identify one or more genes for which the expression levels in submatrix B substantially differ from the expression levels in submatrix A. For example, a gene showing greater expression levels in submatrix A than in submatrix B may be considered to be up-regulated by a particular event, while a gene showing greater expression levels in submatrix B than in submatrix A may be considered to be down-regulated.

In order to determine whether or not the measured expression levels for a particular gene are different in submatrices B and A, various different approaches are currently employed. In a very simple approach, the average of the measured expression levels in a row of submatrix B may be compared to the average of the measured expression levels in the corresponding row of submatrix A. However, gene-expression values are generally distributed over a range of values according to one or more probability distributions. Simply comparing average expression values for two different classes may not provide a reliable indication of differential expression, particularly when only relatively small variations in expression levels may be nonetheless significant. One common approach is to assume a normal, or Gaussian, distribution for expression levels. One can then employ the well-known t-test in order to determine, at a desired level of certainty, whether or not the distributions of the expression levels in two different classes represented by submatrix B and submatrix A have different means, and are therefore differentially expressed, or whether the two distributions cannot be determined to have different means, and therefore cannot be determined to be differentially expressed at the desired level of certainty.

FIGS. 4A-D illustrate several different types of expression-level-distribution scenarios. As one example, when the expression-level data for a particular gene i, Eᵢ, is plotted by plotting, with respect to a vertical axis, the number of samples, or experiments, in which the expression level falls in each interval ΔEᵢ, over a domain of expression-level intervals, the expression-level distribution 402 shown in FIG. 4A might be observed. The curve 402 is shown, in FIG. 4A, as continuous, for simplicity of illustration, although it is actually discrete. Similar continuous-appearing hypothetical plots of discrete data is employed in subsequent figures. Separately plotting the expression-level data for the before class B and the after class A, as shown in FIG. 4B, reveals that the data for each class is distributed according to two different normal distributions 404 and 406 with two different, widely separated means 408 and 410, respectively. Thus, the overall gene-expression-level distribution, shown in FIG. 4A, can be resolved into two different, normal distributions 404 and 406 corresponding to the two classes B and A. Similarly, a Gaussian-appearing distribution 412 in FIG. 4C for the combined gene-expression data Eᵢ may be resolved, by separate plotting of expression-level distributions, in FIG. 4D, as two Gaussian distributions 414 and 416 with two different, narrowly separated means 418 and 420. At some point, when the difference in means Δμ falls below a threshold value that depends on the standard deviation for the distributions and on the closeness of fit of the distributions to normal distributions, it cannot be determined, with an adequate level of certainty, whether or not the class-specific distributions represent two different normal distributions with different means, or whether, in fact, the genes are not differentially expressed and the corresponding gene-expression-level values are distributed according to a common probability distribution.

The t-test computes a t-statistic from the means and standard deviations for the expression data for two classes as follows:

$$\frac{{μ}_{1} - {μ}_{2}}{{σ}_{ɛ}} = \frac{{\overline{x}}_{1} - {\overline{x}}_{2}}{\sqrt{\frac{( {n}_{1} - 1 ) {S}_{1}^{2} + ( {n}_{2} - 1 ) {S}_{2}^{2}}{{n}_{1} + {n}_{2} - 2}} ( \frac{1}{{n}_{1}} + \frac{1}{{n}_{2}} )}$$

where
\overline{x}₁ is the average expression level for class 1;
\overline{x}₂ is the average expression level for class 2;
S₁ is the sample variance for class 1;
S₂ is the sample variance for class 2;
n₁ is the number of expression level data for class 1;
n₂ is the number of expression level data for class 2; and
σ_{ε} is related to the pooled variance.
   The computed t-statistic is compared to tables of t-statistics that tabulate critical t values for different significance levels. When the computed t value exceeds the critical t value for a particular significance level, then it can be concluded that the expression-level distributions for the two classes have distinct means, and that the gene is differentially expressed. The t-test is an example of a parametric test for differential expression. A parametric test assumes a particular type of gene-expression-level distribution. In the case of the t-test, a normal distribution is assumed. The t-test has a great advantage in providing a significance level associated with each differential-gene-expression-level determination. In other words, a numerical confidence in any particular differential-gene-expression-level determination, such as a p-value, can be computed along with the particular differential-gene-expression determination. The numerical confidence values may be used, in turn, to prioritize differential-gene-expression determinations, to distinguish genes that can be classified as differentially expressed with respect to a particular event with high confidence from those which seem to show differential expression, but for which the differential-expression indication is of relatively low significance. Unfortunately, gene-expression-level distributions are infrequently normal, and the t-test is therefore often either inapplicable, or insufficiently accurate.

When the expression-level distributions are unknown, non-parametric tests may be employed. One example is the Wilcoxon ranked-sum test. FIGS. 5 and 6A-E illustrate the general approach taken by the Wilcoxon ranked-sum test method in the context of the two-class example discussed with reference to FIG. 3. In a first step, shown in FIG. 5, the expression-level data for all experiments with respect to gene i, Eᵢ 502, and the expression-level data for gene i for classes B 504 and A 506, are sorted in ascending order of expression-level value to produce corresponding sorted expression-level data sets Eᵢ^{s} 508, Bᵢ^{s} 510, and Aᵢ^{s} 512. The indices for the sorted data arrays Eᵢ^{s}, Bᵢ^{s}, and Aᵢ^{s} can be thought of as ranks, with the expression levels ranked in ascending expression-level value. Next, as shown in FIG. 6A, the expression levels are plotted against the corresponding ranks, to produce an expression-level/rank curve 602. In FIGS. 6B and 6D, expression-level/rank curves are plotted separately for the sorted class B data, Bᵢ^{s}, and in FIGS. 6C and 6E, expression-level/rank curves are plotted for the sorted class A data Aᵢ^{s}. FIGS. 6B and 6C together represent an extreme case in which gene i is unambiguously differentially expressed in class B and in class A, and is, in fact, up-regulated. Note that, if the expression-level/rank curves of FIGS. 6B and 6C are joined together, with the end point of the curve of FIG. 6B joined to the starting point of the expression-level/rank curve of FIG. 6C, the expression-level/rank curve 602 in FIG. 6A is exactly produced. This indicates that all of the expression levels in Bᵢ^{s} reside in the lower portion of the overall expression-level/rank curve 602 in FIG. 6A, while the expression levels in As all reside in the upper portion of the expression-level/rank curve 602 of FIG. 6A. In other words, all of the expression-level values of class B are lower than all of the expression-level values of class A, unambiguously indicating that the overall expression level for gene i in class B is lower than the overall expression level for gene i in class A. Thus, the hypothetical results represented by FIGS. 6A-C strongly indicate differential expression, and represent an extreme case of expression-level-distribution resolution via the Wilcoxon rank-sum test. In contrast, the separately plotted expression-level/rank curves in FIGS. 6D and 6E represent a case of non-differential expression. The expression-level/rank curves in FIG. 6D and FIG. 6E are quite similar, with similar slopes, starting points, and end points. In fact, they represent horizontally compressed versions of the expression-level/rank curve 602 in FIG. 6A. This indicates that the expression levels of class B and class A significantly overlap in distribution.

In a particular type of Wilcoxon test, the signed-rank test, the values for differences in expression for sample sources with respect to an event are computed. The absolute values of the computed differences are ranked, and the ranks are then signed according to the signs of the originally computed differences. The signed ranks are then summed to produce the sum W. When repeatedly computed for large numbers N of computed differences, W is normally distributed with mean μw=0 and standard deviation

$$σ = \sqrt{\frac{( N + 1 ) ( 2 N + 1 )}{6}} .$$

Therefore, a z-ratio can be computed for a particular W value, where

$$z = \frac{W - 0.5}{{σ}_{W}} ,$$

and the computed z-ratio can be compared to critical z-ratio values for a particular N to determine a level of significance within which a null hypothesis can be rejected or accepted.

Many other, additional, nonparametric tests are currently employed, including the Kolmogorov-Smirnov score, the information score, and the threshold-number-of misclassifications (“TNoM”) method. Indications of differential expression produced by the nonparametric tests often do not correspond, in magnitude, to the usefulness of differential expression of genes from a biological standpoint. For example, according to the Wilcoxon rank-sum test, a gene that is always, but only very slightly, up-regulated is assigned a higher score than a gene that is almost always, but highly, up-regulated with a few exceptional cases of slight down-regulation.

Non-parametric tests are, however, extremely useful and necessary in gene-expression analyses, because often gene-expression analyses involve relatively small sample sizes, leading to low-significance results, and because patient-specific variability often masks general gene-expression-level trends. FIGS. 7A-C illustrate the inherent shortcomings of parametric tests. In FIG. 7A, the before and after data for 6 patients j₁-j₆ with respect to gene i are shown in the row vectors i_{B} 702 and i_{A} 704, and the differences between the expression levels for gene i for each patient are shown in row vector i_{Δ} 706. In FIG. 7B, normal curves 708 and 710 are fit to the data in vectors i_{B} and i_{A}, respectively, with the data points for each vector plotted as dots, below, with arrows, such as arrow 712, showing the change for each patient. Although (as can be seen in FIG. 7A from the data values in vectors i_{B} and i_{A}, and in FIG. 7B by the directions of the arrows below the plotted, fitted normal curves) gene expression levels increase for 5 out of 6 patients, a t-test of the before and after data does not indicate differential expression with a reasonably low p-value, or, in other words, with a reasonably high level of confidence. This is due to significant overlap between the before and after distributions, resulting from rather marked variability in the expression levels for individual patients. In FIG. 7C, the per-patient changes in gene expression contained in vector i_{Δ} are plotted, from which it can be readily observed that the distribution of gene-expression changes is decidedly non-normal. Therefore, unless a well-defined probability distribution can be inferred from the data, a matched parametric test based on an assumed normal distribution also does not produce an indication of gene expression elevation with reasonable confidence. In cases such as that illustrated in FIGS. 7A-C, a non-parametric test is needed.

Because identifying genes that are differentially expressed with respect to different types of events has become so important for researchers, diagnosticians, clinicians, and other professionals, techniques for facilitating identification of such differentially expressed genes are actively and enthusiastically sought. In particular, since the assumptions on which the t-test is based are infrequently encountered in gene-expression data, and since inter-patient variability often obscures significant gene-expression trends, it would be desirable to identify non-parametric tests for differential expression that produce scores with magnitudes reflective of practical and biological usefulness and that emphasize general gene-expression trends despite variability in sample sources. Importantly, it is particularly desirable that such non-parametric tests for differential expression produce, in addition to scores with magnitudes reflective of practical and biological usefulness, numerical significance levels associated with the scores, to allow for scientific prioritization of genes determined to be differentially expressed by the confidence of the determination.

### SUMMARY OF THE INVENTION

In various embodiments of the present invention, initial experimental data is initially partitioned into classes by sample source, concentration or number-of-molecule values are computed with respect to each initial partition, and a rank consistency score or fold-change consistency score is computed for various molecular concentration or number-of-copies determinants with respect to one or more class-specifying events of interest. In other words, rather than partitioning experimental data directly into two or more classes relative to an event of interest, the experimental data is first partitioned according to sample source, and then each sample-source partition is partitioned into two or more classes relative to an event of interest. In various specific embodiments of the present invention, initial gene-expression data is initially partitioned into classes by patient, subject, or other identifier of a source of samples, expression-level-differences are computed for each gene with respect to each initial partition, and a rank consistency score or fold-change consistency score is computed for each gene from the expression-level difference metrics computed for each initial partition. Rank-consistency and fold-change-consistency scores may be calculated for each gene of interest, along with levels of significance, or p-values, for the rank-consistency scores and fold-change consistency scores.

### BRIEF DESCRIPTION OF THE DRAWINGS

FIG. 1 shows a matrix representation of a gene-expression data set.

FIG. 2 illustrates partitioning of gene-expression data matrix E into two submatrices, or classes.

FIG. 3 illustrates the gene-expression data for a particular gene contained within the initial gene-expression-data matrix E and within submatrices B and A.

FIGS. 4A-D illustrate several different types of expression-level-distribution scenarios.

FIGS. 5 and 6A-E illustrate the Wilcoxon ranked-sum test method in the context of the two-class example discussed with reference to FIG. 3.

FIGS. 7A-C illustrate the inherent shortcomings of parametric tests.

FIGS. 8A-B illustrate first and second sample-source partitioning steps common to embodiments of the present invention.

FIG. 9 illustrates a third step common to various embodiments of the present invention.

FIGS. 10-11 illustrate computation of rank consistency scores from the gene-expression-difference column vectors computed as illustrated in FIG. 9.

FIG. 12 illustrates a second embodiment of the present invention.

FIG. 13 illustrates a cumulative distribution function.

FIG. 14 provides a control flow diagram for the rank consistency and fold-change consistency scores.

FIG. 15 shows an example of heatmap visualization of the difference-metric display matrix D.

FIG. 16 shows another example of visualization of the same display matrix D as in FIG. 15, using a different mapping between colors and between Dₖ(i) value magnitudes.

### DETAILED DESCRIPTION OF THE INVENTION

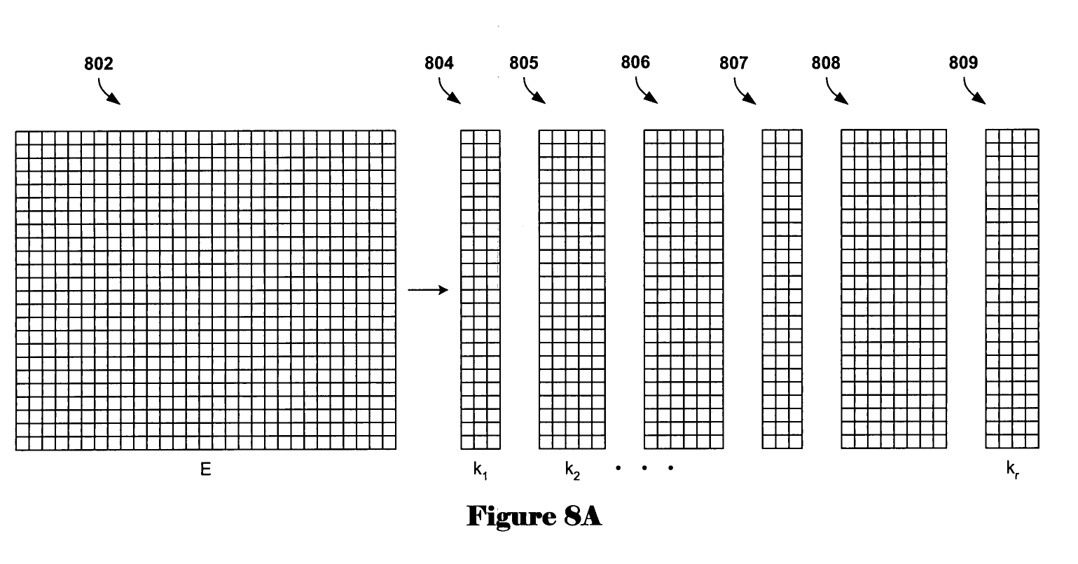

In various embodiments of the present invention, gene-expression data is partitioned first according to sample source, and then, within each sample-source partition, again partitioned with respect to two or more classes relative to one or more events. By first partitioning gene-expression data with respect to sample source, additional, valuable information related to the inherent self-controlled characteristic of expression-level data obtained from a single source can be recovered and used to produce differential expression scores more reflective of the biological and practical significance of detected differential expression. Gene-expression levels may vary considerably between sample sources, such as between patients in a medical study, in ways that can obscure general, differential gene-expression trends within the data. Measured gene-expression levels for a particular patient or sample source generally exhibit less variation, and are, in a sense, self-controlled. Therefore, gene-expression-level differences observed in gene-expression data for a particular patient or sample source may have greater significance than observed, general gene-expression-level differences between arbitrary samples or experiments. The first partitioning of gene-expression data with respect to sample source allows for gene-expression-level differences for each patient or sample source to be detected. In various embodiments of the present invention, rank-based consistency scores are computed for each gene as a determination of the differential expression of the gene with respect to one or more events, and, importantly, a significance value, or p-value, is computed and associated with each rank-based consistency score. The embodiments of the present invention are discussed, below, with reference to FIGS. 7-13.

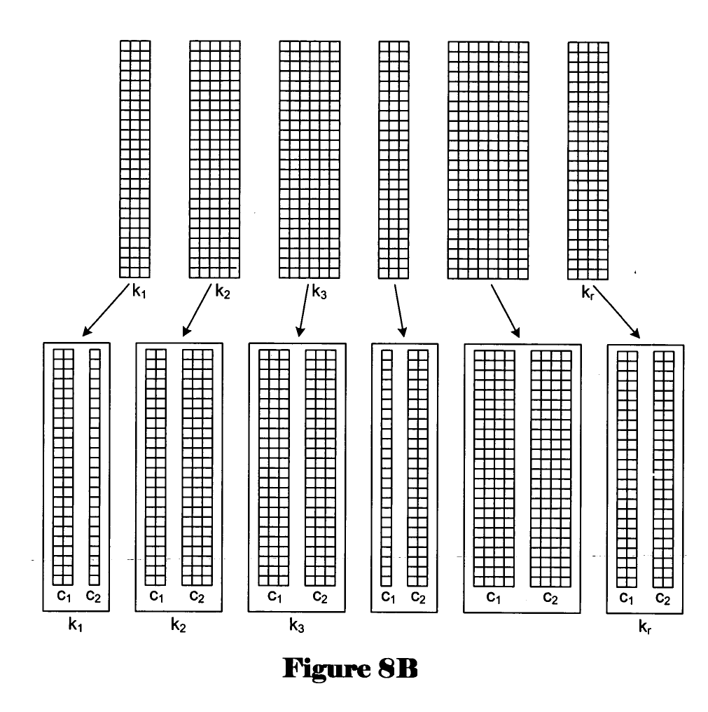

FIGS. 8A-B illustrate first and second sample-source partitioning steps common to embodiments of the present invention. As shown in FIG. 8A, a rectangular matrix E 802 representing an initial gene-expression-level data set is partitioned into submatrices K₁, K₂, . . . Kᵣ 804-809 with respect to r different sample sources k₁, k₂, . . . , kᵣ. For example, in a medical research project analyzing gene expression in a number of different patients, the combined gene-expression-level data is partitioned, as shown in FIG. 8A, into partitions each corresponding to a different patient.

FIG. 8B illustrates a second step common to various embodiments of the present invention. As shown in FIG. 8, each of the sample-source partitions K₁, K₂, . . . Kᵣ are each further divided into two or more classes. In the example shown in FIG. 8B, each sample-source partition is divided into two classes C₁ and C₂. This second partitioning may be explicit, or may be implicitly carried out in computing of expression-level-difference metrics.

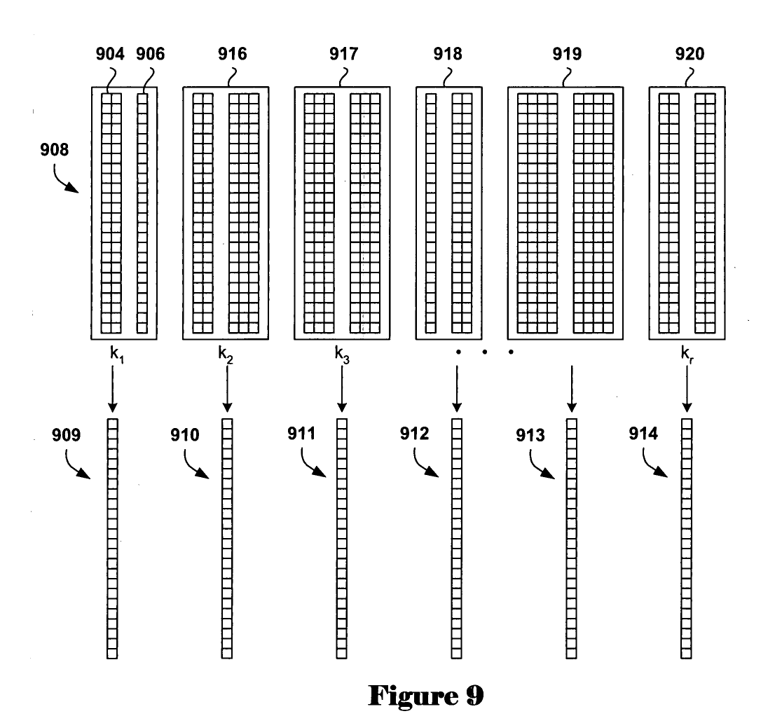

FIG. 9 illustrates a third step common to various embodiments of the present invention. For each sample-source partition K₁-KR, a gene-expression-level-difference metric is computed for each gene and stored in a column vector. For example, as shown in FIG. 9, a column vector 902 of gene-expression-difference metrics is computed from class C₁ 904 and class C₂ 906 of initial, sample-source partition 908. Distinct gene-expression-level-difference column vectors 910-914 are computed from each of the other, initial, sample-source partitions K₁-Kᵣ 916-920. Calculation of gene-expression-difference metrics is discussed below. It should be noted that any of many different gene-expression-difference metrics may be employed in the various embodiments of the present invention.

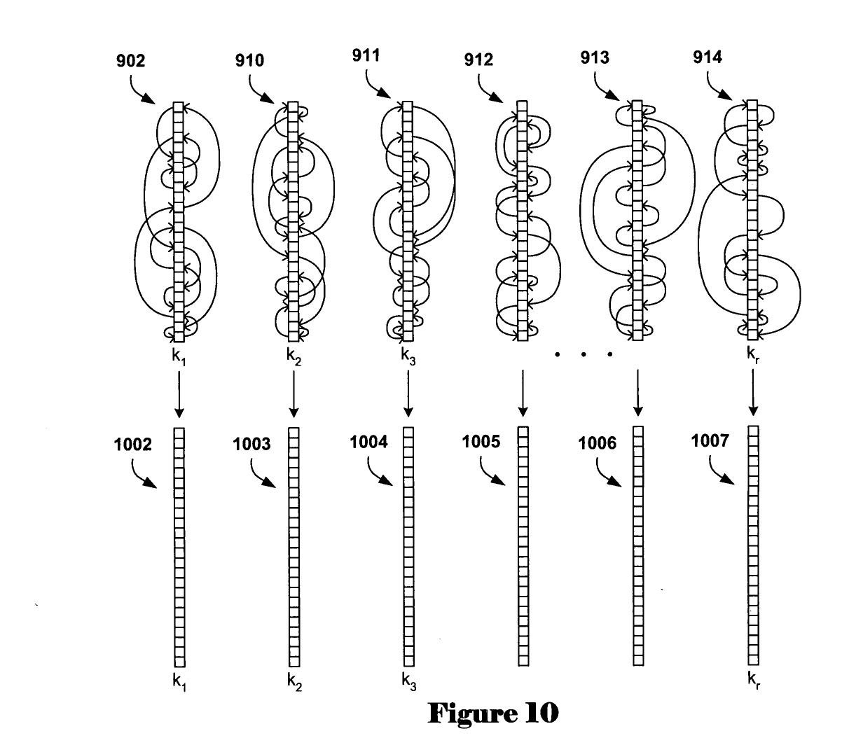

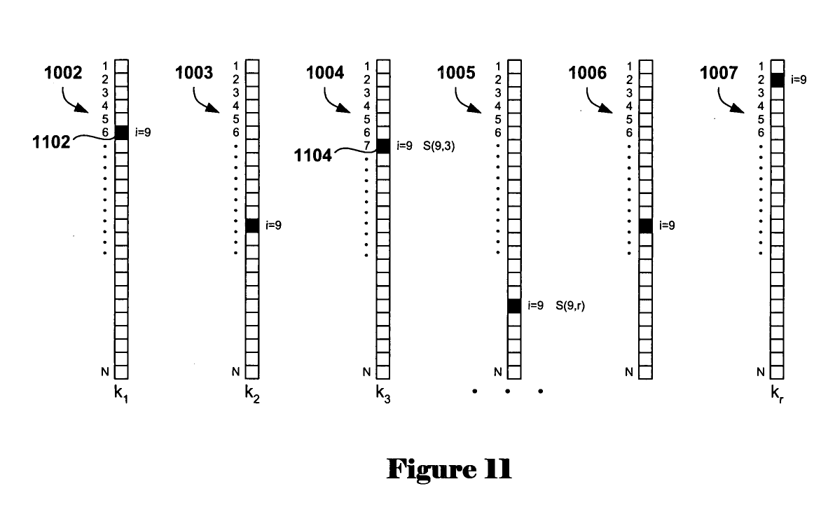

A first embodiment of the present invention involves computation of rank consistency scores (“RCoSs”). FIGS. 10-11 illustrate computation of rank consistency scores from the gene-expression-level-difference column vectors computed as illustrated in FIG. 9. In FIG. 10, each of the initially produced gene-expression-level-difference column vectors 902 and 910-914 are sorted, by difference metrics, to produce sorted difference column vectors, or rank vectors 1002-1007, respectively. FIG. 11 shows computation of a particular rank consistency score for a particular gene from the rank column vectors computed as shown in FIG. 10. An RCoS, denoted s(g;m), is computed for a particular gene g based on m out of r source samples. FIG. 11 shows the ranks computed for the gene i=9 (g=9) in the different rank column vectors 1002-1007. For example, gene 9 has difference rank 6 1102 in the difference-rank column vector corresponding to sample-source k₁. To compute the RCoSs s(9;3), the third smallest rank, or third largest gene-expression-level-difference metric, observed for gene 9 in all of the difference-rank column vectors 1104 is chosen. The value of s(9;3) is the third smallest rank, or, in the example shown in FIG. 11, 7, divided by the total number of genes N. The RCoSs s(g;r) is a special case of the general RCoS s(g;m), representing the largest rank, or smallest observed gene-expression-level difference, for a particular gene observed in any of the difference-rank column vectors divided by N. Note that s(g;r)N is the largest rank, or smallest gene-expression difference, observed for gene g in all of the different sample-source partitions.

An RCoS thus may accurately reflect the practical and biological significance of differential gene expression. For example, considering the hypothetical situation illustrated in FIG. 11, if the s(g;m) value, computed for gene g with m/r close to 1.0, is relatively low, then gene g shows a high degree of differential expression in nearly all sample sources, or patients in the case of human data. In other words, gene g is in the top s(g;m)-th fractions for differentially expressed genes on a sample-source-by-sample-source basis. Therefore, the RCoS reflects the level of consistent and large-magnitude differential expression within self-controlled data sets, and is more reflective of practical and biologically significant differential gene expression than differential gene-expression metrics computed from class partitions of an initial, combined, gene-expression-level data set.

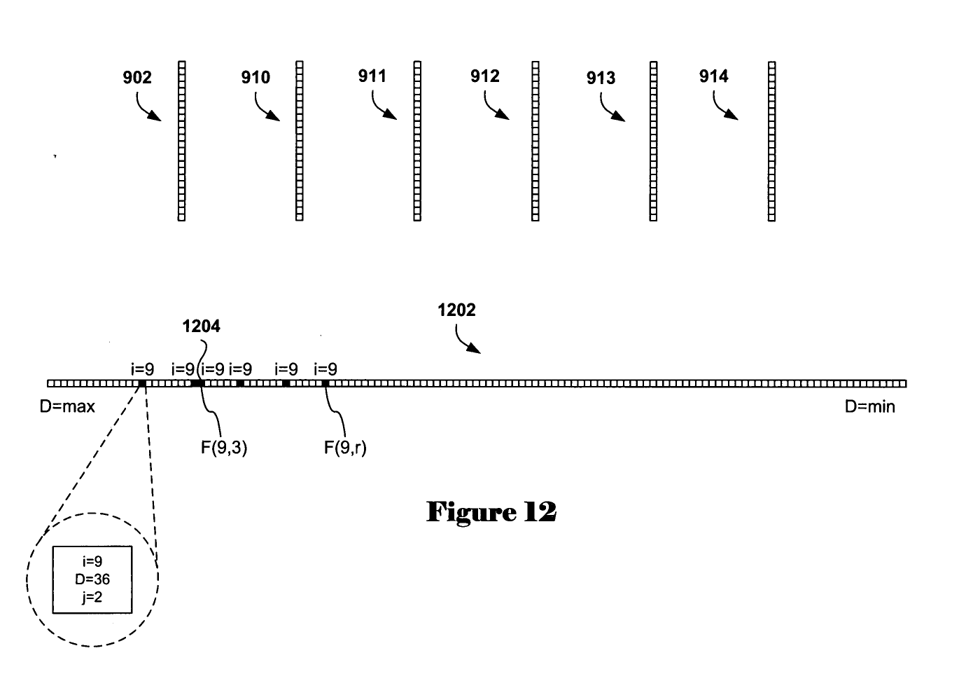

FIG. 12 illustrates a second embodiment of the present invention. In FIG. 12, computation of a fold-change consistency score (“FoCoS”) from the difference-rank column vectors computed as illustrated in FIG. 9 is illustrated. The difference-score column vectors 902 and 910-914 are combined and sorted with respect to difference-metric values in order to produce a pooled difference-metric vector 1202. From the sorted, difference metric vector 1202, FoCoS scores (g;m) are computed for a gene g based on m out of r (m/r) sample sources from the sorted, difference-metric vector 1202 in a manner similar to computation of the RCoS scores, shown in FIG. 11. For example, to compute f(9;3), the entry 1204 containing the third largest difference-metric for gene 9 is found in the sorted, difference-metric vector 1202. The FoCoS f(9;3) is the difference metric contained in entry 1204. Note that, in the difference-metric-vector 1202, entries may contain indications of the gene, sample, and computed difference metric i, j, and Dₖ(i), respectively. The difference-metric vector may be sorted by gene as well as by difference-metric value, to facilitate computing FoCoS scores. As with RCoS scores, the FoCoS score f(g;r) is a special case representing the smallest difference metric observed in the data for gene g.

With the overall method of computing RCoS and FoCoS values described, above, the mathematical details for specific calculations of RcoS and FoCoS scores can be next provided. First, expression-level differences that can be employed in computation of both RCoS and FoCoS scores are computed in one of several possible ways. For one embodiment, statistical parameters are first calculated:

$${μ}_{k , 1} ( i ) = \frac{1}{\vert {C}_{1} \vert} {\sum }_{j ∈ {C}_{k , 1}} {E}_{i , j}^{k}$$

$${μ}_{k , 2} ( i ) = \frac{1}{\vert {C}_{2} \vert} {\sum }_{j ∈ {C}_{k , 2}} {E}_{i , j}^{k}$$

$${σ}_{k , 1} ( i ) = {( \frac{1}{\vert {C}_{1} \vert} {\sum }_{j ∈ {C}_{k , 1}} {( {E}_{i , j}^{k} - {μ}_{k , 1} ( i ) )}^{2} )}^{1 / 2}$$

$${σ}_{k , 2} ( i ) = {( \frac{1}{\vert C2 \vert} {\sum }_{j ∈ {C}_{k , 2}} {( {E}_{i , j}^{k} - {μ}_{k , 2} ( i ) )}^{2} )}^{1 / 2}$$

where
   |C₁| is the number of gene-expression-level values in class 1;
   |C₁| is the number of gene-expression-level values in class 2;
E_{i,j}^{k} is the log of the gene-expression-level value determined for gene i in sample j;
C_{k,1} is the class 1 partition of sample-source partition k;
C_{k,2} is the class 2 partition of sample-source partition k;
μ_{k,1} is the mean gene-expression log value for the class 1 partition of sample-source partition k;
μ_{k,2} is the mean gene-expression log value for the class 2 partition of sample-source partition k;
σ_{k,1} is the variance for gene-expression log values of the class 1 partition of sample-source partition k; and
σ_{k,2} is the variance for gene-expression log values of the class 2 partition of sample-source partition k.
Dₖ(i), the difference metric for gene i computed for sample source partition k, is then given by:
Dₖ(i)=μ_{k,1}(i)−μ_{k,2}(i)
or by

$${D}_{k} ( i ) = \frac{{μ}_{k , 1} ( i ) - {μ}_{k , 2} ( i )}{{σ}_{k , 1} ( i ) + {σ}_{k , 2} ( i )}$$

These are but two of many different possible difference metrics that may be employed. Other possibilities include difference metrics based on the Gaussian error score, t-test, Info, and TNoM, among others.

Of great importance is the computation of p-values, or significance values, for RCoS and FoCoS scores. For the RCoS score s(g;m), the p-value p-Val(s,m) is given by:

$$p - Val ( s , m ) = {\sum }_{k = m}^{r} ( \begin{matrix}r \\ k\end{matrix} ) {{s}^{k} ( 1 - s )}^{( r - k )}$$

where
r is the number of sample sources;
k is a particular sample source; and
s is s(g;m).
   The p-value p-Val(s,m) is essentially the probability of finding m sample sources out of r sample sources with RCoS values in the top s fraction of all ranks in all sample-source partitions for randomly generated ranks. In other words, if V is an r-dimensional random vector containing values drawn independently and uniformly from {1, . . . , N}, then p-Val(s,m) is the probability of the m^{th} smallest entry in V being smaller than sN. Using the computed p-values, the false discovery rate (“FDR”) and binomial surprise rate (“BSR”) can be computed for a differentially expressed gene with RCoS scores equal to or better than s. For a given RCoS score s, set p=p-Val(s,m), assuming that m and r are fixed. Determine the number of genes with m/r RCoS values better than s as n(s). The BSR is defined as:
−log(σ(s))
where σ(s)=probability that N(s)≧n(s)

$$= {\sum }_{k = n ( s )}^{N} ( \begin{matrix}N \\ k\end{matrix} ) {{p}^{k} ( 1 - p )}^{( N - k )}$$

and the FDR is defined as:
pN/n(s)

Note that the BSR has a high value for genes with significant differential gene expression, and when plotted with respect to RCoS, often shows a peak corresponding to the most significantly differentially expressed genes. The FDR is essentially the ratio of expected to observed genes with RCoS scores equal to or better than s.

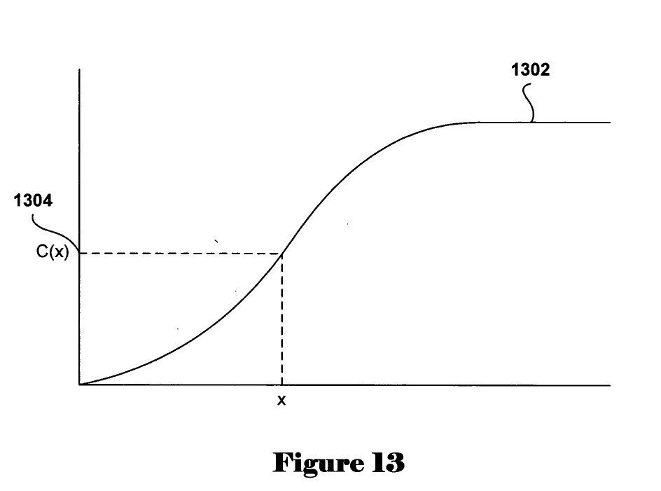

FIG. 13 illustrates a cumulative distribution function. The cumulative distribution function C(x) 1302 gives, for a particular random-variable value x, a computed y-value 1304 C(x) representing the probability that a random-variable would have a value less than the random-variable value x at any particular one of a number of sampling points. There is a cumulative distribution function that corresponds to each different probability distribution.

For the FoCoS scores, p-values can be computed from a distribution of the difference metrics Dₖ(g). In one variation, the difference-metric values can be considered to be normally distributed, with mean μ and standard deviation σ given by:

$$μ = \frac{1}{Nr} {\sum }_{i , k} {D}_{k} ( i )$$

$$σ = \sqrt{\frac{1}{Nr} {\sum }_{i , k} {( {D}_{k} ( i ) - μ )}^{2}}$$

The p-value for gene g with m/r FoCoS value f(g;m) is a probability of drawing r independent numbers according to the cumulative distribution function C(x) for the normal distribution of difference metrics and obtaining m values larger than f(g;m), or, in other words, the p-value for the FoCoS metric f(g;m) is given by:

$$p - Val ( f ; m ) = {\sum }_{k = m}^{r} ( \begin{matrix}r \\ k\end{matrix} ) {( 1 - C ( f ) )}^{k} {C ( f )}^{( r - k )}$$

Rather than using an assumed Gaussian cumulative distribution function C(x), an observed cumulative distribution function F(x) can be employed, where F(x) is defined as:

$$F ( x ) = \frac{\vert \{ ( g , k ) \text{:} {D}_{k} ( g ) \leq x \} \vert}{Nr}$$

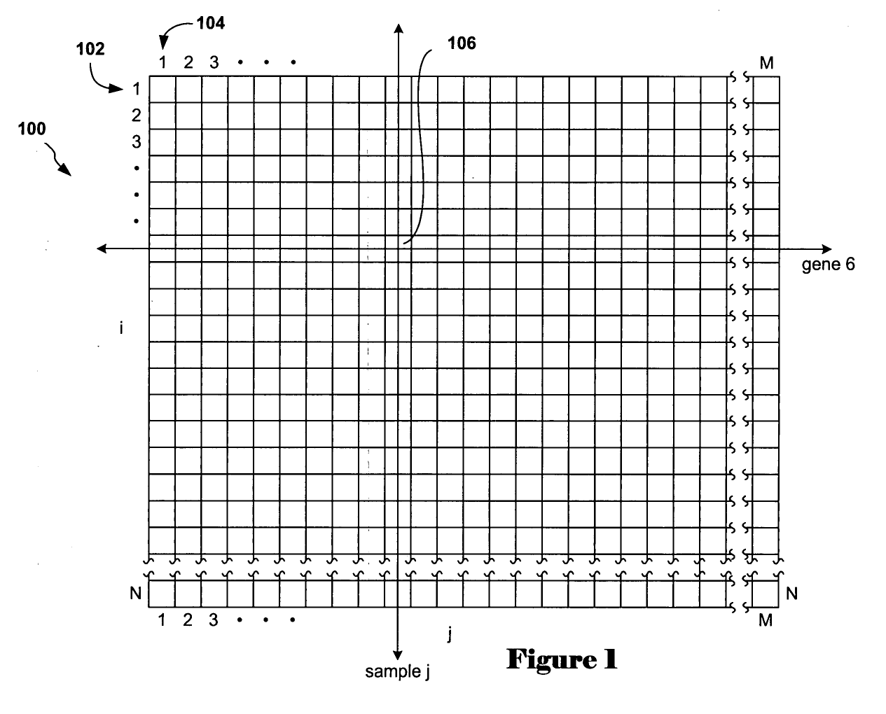

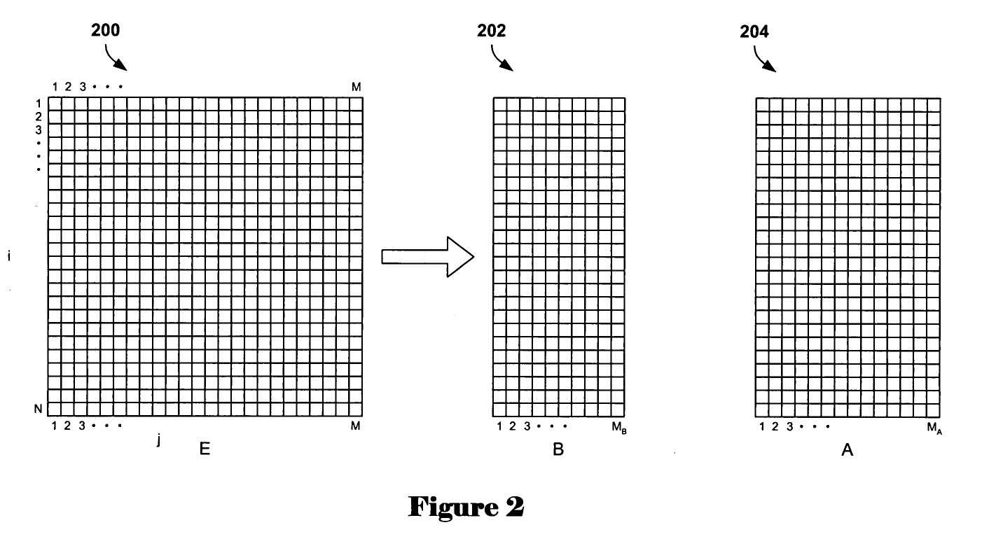

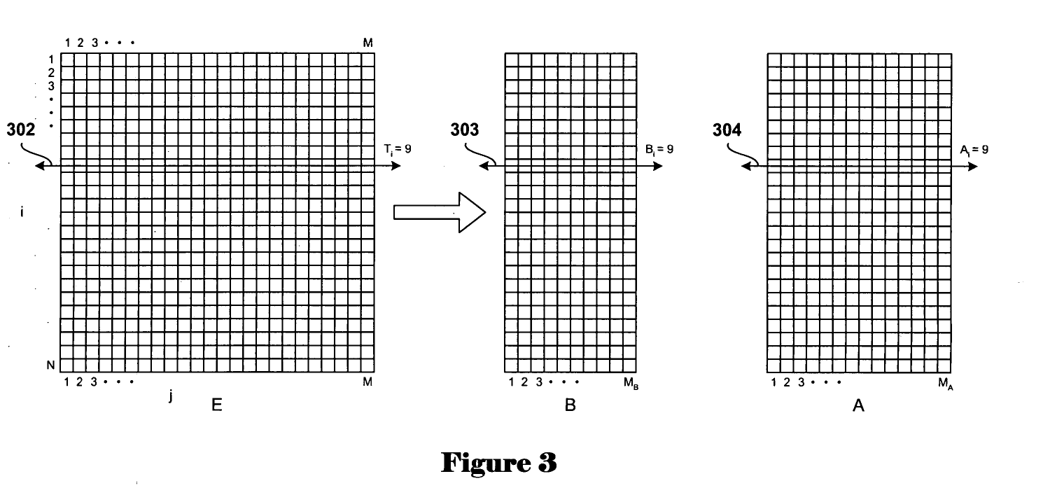

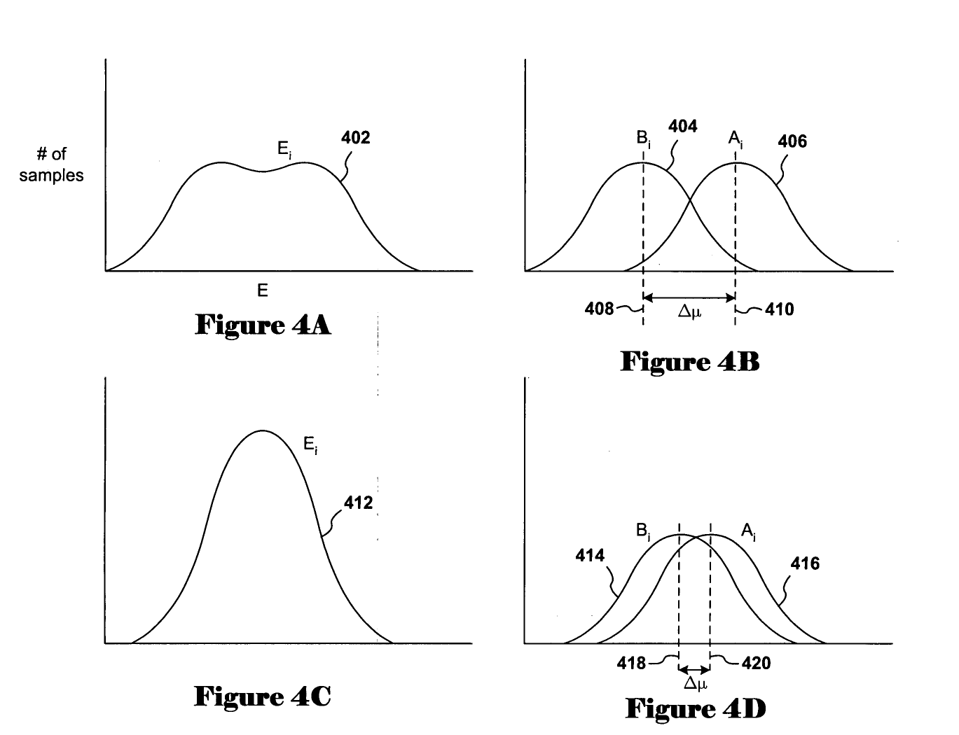

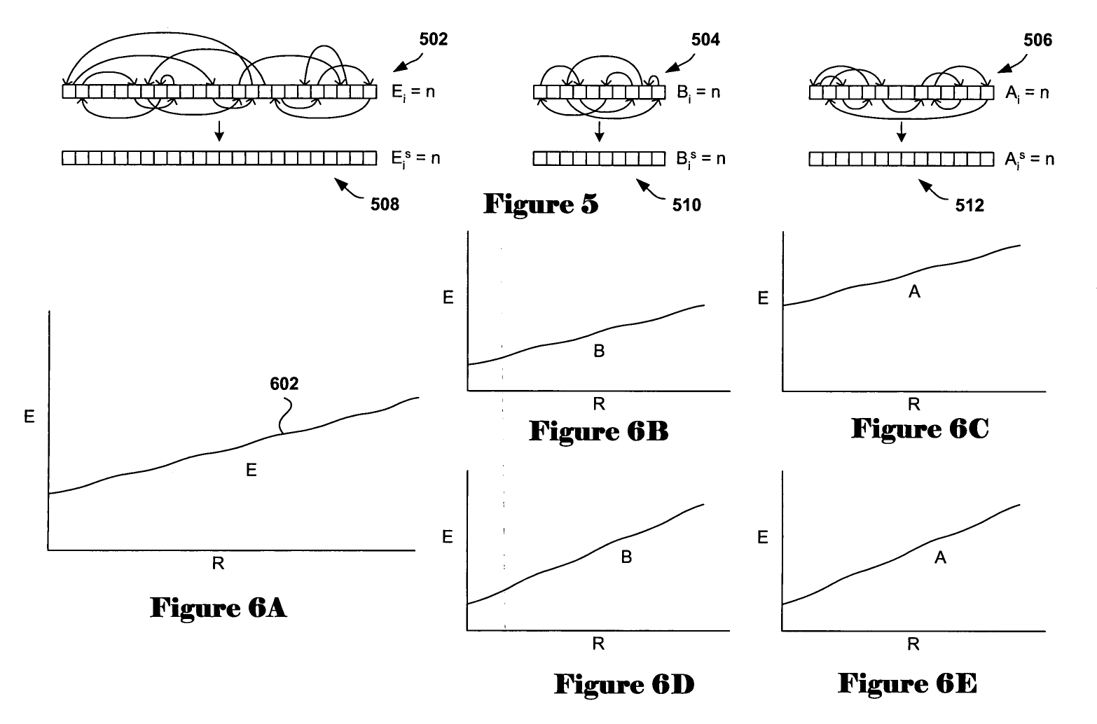

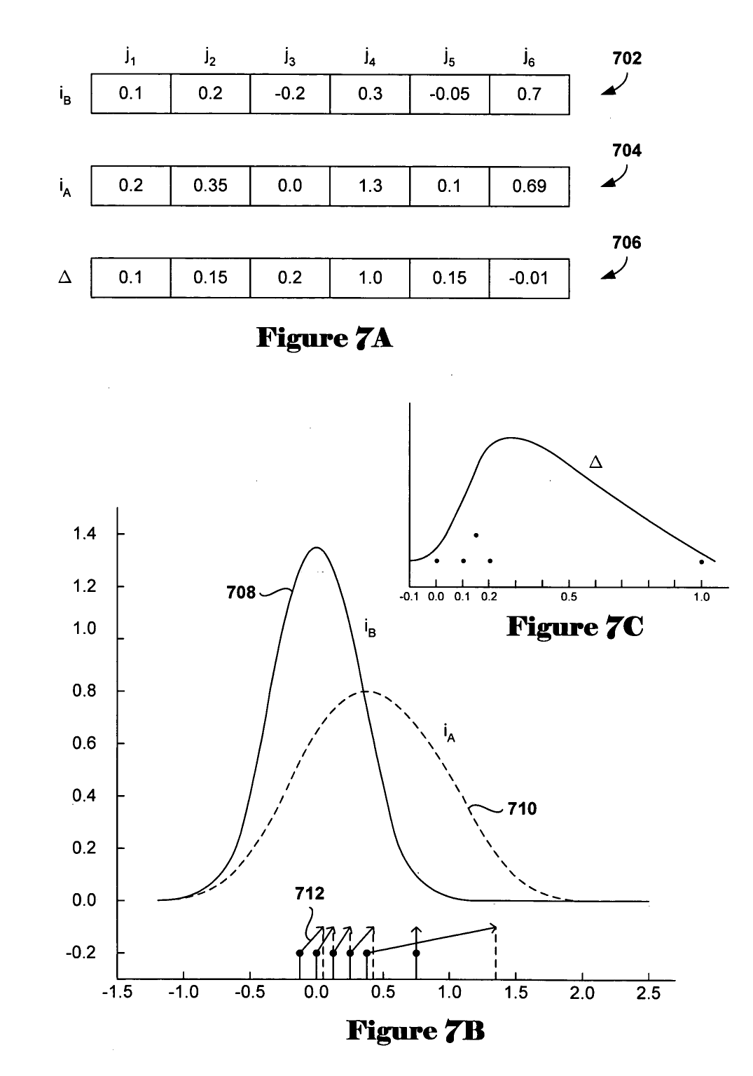

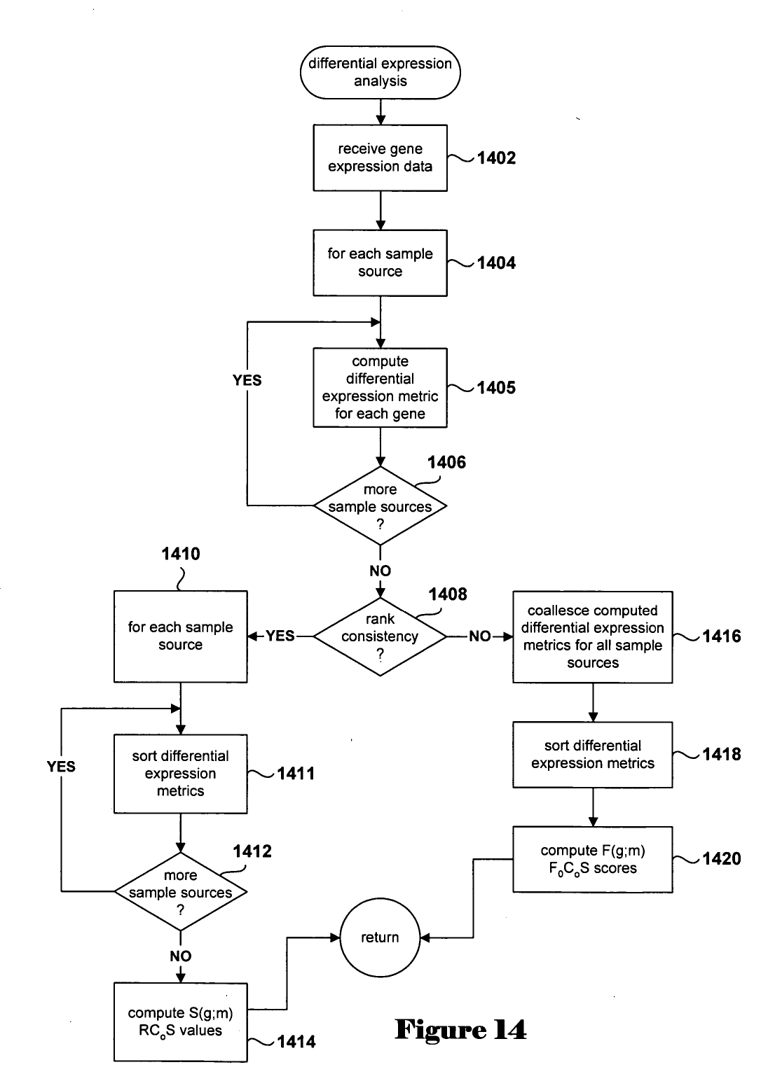

FIG. 14 provides a control flow diagram for the rank consistency and fold-change consistency scores. FIG. 14 summarizes various sample-source-enhanced differential gene-expression scores computed by embodiments of the present invention. First, in step 1402, gene-expression data, such as gene-expression data represented by gene-expression-data matrix 100 in FIG. 1, is received. Next, in the for-loop of steps 1404-1406, for each sample-source, a difference metric is computed for each gene, as illustrated in FIGS. 7-9. If rank-consistency scores are desired, as determined in step 1408, the difference-metric vector for each sample source is sorted, as illustrated in FIG. 10, in the for-loop of steps 1420-1412, and rank-consistency scores are computed in step 1414, as illustrated in FIG. 11. On the other hand, if fold-change consistency scores are desired, as determined in step 1408, then the difference-metric vectors for all sample sources are pooled, in step 1416, the pooled vector is sorted, in step 1418, and fold-consistency scores are computed in step 1420 as illustrated in FIG. 12.

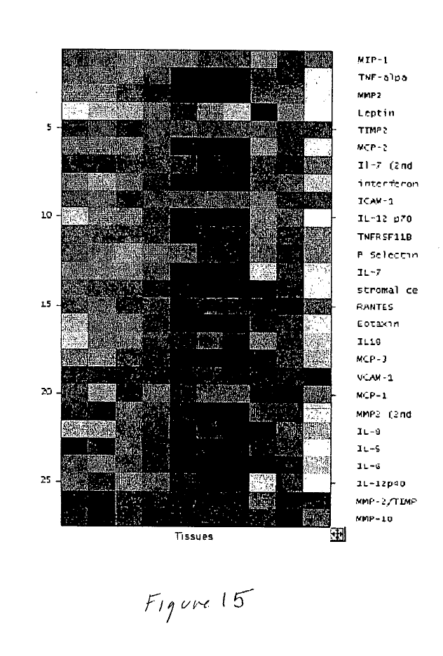

A useful, visual representation of difference metrics Dₖ(i) for each sample source k and each gene i may be obtained as follows. Each cell of a displayed matrix D representing difference metrics Dₖ(i), with row index i and column index k, can be displayed in a color representative of the magnitude of Dₖ(i). For example, a darkest color (e.g. black, or blue) may correspond to a smallest magnitude of Dₖ(i) and a lightest color (e.g. white, or yellow) may correspond to a largest magnitude of Dₖ(i), with cells representing Dₖ(i) values with intermediate magnitudes represented by mixtures of the darkest color and the lightest color in a ratio corresponding to the relative magnitude of the intermediate-magnitude Dₖ(i) values (e.g. shades of gray, or mixtures of blue and yellow). Many other mappings between Dₖ(i) value magnitudes and colors are possible. A dependency between intensity of color of representation of, and the value of, a difference metric can be modeled using various monotone functions, such as by a linear function, as in example provided in FIGS. 14 and 15.

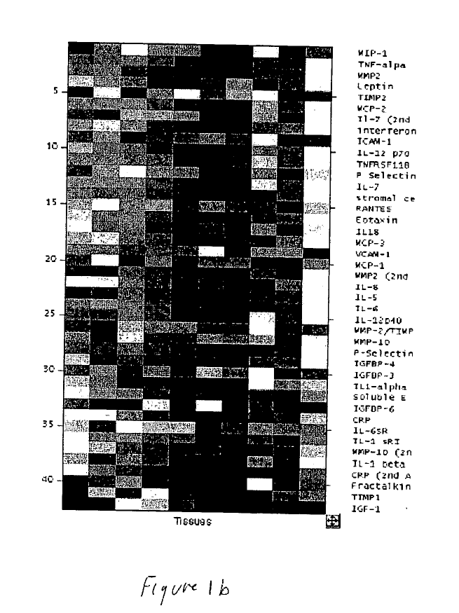

In a heatmap representation of difference metrics Dₖ(i), each row correspond to changes in a gene expression level, protein level, metabolite level, or other concentration or molecular abundance, and each column represent a different sample source. Genes maybe be sorted by RCoS, or by FoCoS, so that the top rows of the heatmap correspond to genes with the most consistent changes. Also, columns may be sorted using properties of sample sources to highlight dependencies of properties of samples to magnitudes of difference metrics. FIG. 15 shows an example of heatmap visualization of the difference-metric display matrix D. The Dₖ(i) values are computed according to the first Dₖ(i) expression, provided above. Genes are sorted by RCoS, so that the genes with the most consistent changes are on the top of the heatmap. The darkest color corresponds to the Dₖ(i) value magnitude, and the lightest color corresponds to the largest Dₖ(i) value magnitude. FIG. 16 shows another example of visualization of the same display matrix D as in FIG. 15, using a different mapping between colors and between Dₖ(i) value magnitudes. In this example, the colors are scaled from darkest to lightest for each gene, or row, of display matrix D, rather than for all for all cells of D, as in FIG. 15.

Although the present invention has been described in terms of a particular embodiment, it is not intended that the invention be limited to this embodiment. Modifications within the spirit of the invention will be apparent to those skilled in the art. For example, as discussed above, any number of different gene-expression-difference metrics may be employed in computation of RCoS and FoCoS scores. Gene expression data may be received and stored in any of an almost limitless number of different forms, and an almost limitless number of different software routines or programs may be devised in accordance with the present invention, including programs that vary in modular organization, language of implementation, control structures, data structures, and other parameters, to compute rank-consistency and fold-consistency differential gene-expression scores. Methods of the present invention may also be embodied in firmware or hardware. Sample-source data may be explicitly partitioned, or may be implicitly partitioned during difference metric computation. As discussed above, the present invention is widely applicable to biological and chemical experimental data in which molecular-abundance determinates show differential responses to one or more events. For example, the method of the present invention may be applied to determining different metabolite products or ratios resulting from particular mutations to a particular protein catalyst, or to quantify the effects of gene-regulating entities.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for determining, from experimental data, a degree to which one or more determinants of molecular abundance of one or more molecules in sample solutions exhibit a differential response with respect to an event, the method comprising:
for each sample source,
computing a difference-metric for a number of determinants;
employing the computed difference-metrics to compute a rank-based consistency score for one or more determinants, each consistency score reflective of the degree to which a determinant exhibits a differential response with respect to the event; and
computing a significance level for each consistency score.

2. The method of claim 1 wherein employing the computed difference-metrics to compute a consistency score for one or more determinants further includes:
sorting r vectors containing the computed difference-metrics for each sample source by the values of the difference-metrics in descending order to produce r rank vectors;
for each of the one or more determinants,
computing a rank-consistency score s(g;m) as the m^{th} smallest rank for determinant gin the r rank vectors.

3. The method of claim 3 wherein computing a significance level for each consistency score s(g;m) further includes computing p-Val(s,m) by:
$$p - Val ( s , m ) = {\sum }_{k = m}^{r} ( \begin{matrix}r \\ k\end{matrix} ) {{s}^{k} ( 1 - s )}^{( r - k )}$$
where
r is a number of sample sources; and
k is a particular sample source.

4. The method of claim 1 wherein employing the computed difference-metrics to compute a consistency score for one or more determinants further includes:
pooling r vectors containing the computed difference-metrics for each sample source and sorting the pooled difference-metrics to produce a pooled vector;
for each of the one or more determinants,
computing a fold-consistency score f(g;m) as the m^{th} largest difference-metric for determinant g in the pooled vector.

5. The method of claim 4 wherein computing a significance level for each consistency score f(g;m) further includes computing p-Val(s,m) by:
$$p - Val ( f ; m ) = {\sum }_{k = m}^{r} ( \begin{matrix}r \\ k\end{matrix} ) {( 1 - C ( f ) )}^{k} {C ( f )}^{( r - k )}$$
where
r is the number of sample sources;
k is a particular sample source; and
C(f) is a cumulative distribution function for consistency scores f(g;m).

6. The method of claim 5 wherein the cumulative distribution function C(f) corresponds to an assumed normal distribution of the consistency scores f(g;m).

7. The method of claim 5 wherein the cumulative distribution function C(f) is an observed cumulative distribution function for consistency scores f(g;m).

8. Computer instructions that implement the method of claim 1 encoded in a computer readable medium.

9. A method for displaying difference metrics computed by the method of claim 1, the method comprising:
mapping difference metric values to colors; and
displaying computed difference values in a display matrix indexed by determinants and sample sources.

10. A system that determines, from experimental data, a degree to which one or more determinants of molecular abundance of one or more molecules in sample solutions exhibit a differential response with respect to an event, the system comprising:
a receiving-and-storing component that receives experimental data obtained from a number of sample sources, the experimental data including, for each sample source, molecular concentrations of number-of-molecule values prior to and following the event;
a difference-metric-computing component that, for each sample source, computes a difference-metric for a number of determinants; and
a scoring component that employs difference-metrics produced by the difference-metric computing component to compute a rank-based consistency score for one or more determinants, each consistency score reflective of the degree to which a determinant exhibits a differential response with respect to the event, and that computes a significance level for each consistency score.

11. The system of claim 10 further including a display component that displays computed difference metrics by:
mapping difference metric values to colors; and
displaying computed difference values in a display matrix indexed by determinants and sample sources.

12. A method for determining, from gene-expression data, a degree to which one or more genes are differentially expressed with respect to an event, the method comprising:
for each sample source,
computing a difference-metric for a number of genes;
employing the computed difference-metrics to compute a rank-based consistency score for one or more genes, each consistency score reflective of the degree to which a gene is differentially expressed with respect to the event; and
computing a significance level for each consistency score.

13. The method of claim 12 wherein computing a difference-metric for a number of genes further includes computing, for each of the number of genes, Dₖ(i) by:
$${D}_{k} ( i ) = \frac{1}{\vert {C}_{1} \vert} {\sum }_{j ε {C}_{k , 1}} {E}_{i , j}^{k} - \frac{1}{\vert {C}_{2} \vert} {\sum }_{j ε {C}_{k , 2}} {E}_{i , j}^{k}$$
where
Dₖ(i) is the difference metric for gene i computed for sample source k;

|C₁| is a number of gene-expression-level values in class 1;

|C₁| is a number of gene-expression-level values in class 2;
E_{i,j}^{k} is a log of the gene-expression-level value determined for gene i in sample j;
C_{k,1} is a class 1 partition of sample-source partition k; and
C_{k,2} is a class 2 partition of sample-source partition k.

14. The method of claim 12 wherein employing the computed difference-metrics to compute a consistency score for one or more genes further includes:
sorting r vectors containing the computed difference-metrics for each sample source by the values of the difference-metrics in descending order to produce r rank vectors;
for each of the one or more genes,
computing a rank-consistency score s(g;m) as the m^{th} smallest rank for gene g in the r rank vectors.

15. The method of claim 14 wherein computing a significance level for each consistency score s(g;m) further includes computing p-Val(s,m) by:
$$p - Val ( s , m ) = {\sum }_{k = m}^{r} ( \begin{matrix}r \\ k\end{matrix} ) {{s}^{k} ( 1 - s )}^{( r - k )}$$
where
r is a number of sample sources; and
k is a particular sample source.

16. The method of claim 12 wherein employing the computed difference-metrics to compute a consistency score for one or more genes further includes:
pooling r vectors containing the computed difference-metrics for each sample source and sorting the pooled difference-metrics to produce a pooled vector;
for each of the one or more genes,
computing a fold-consistency score f(g;m) as the m^{th} largest difference-metric for gene g in the pooled vector.

17. The method of claim 16 wherein computing a significance level for each consistency score f(g;m) further includes computing p-Val(s,m) by:
$$p - Val ( f ; m ) = {\sum }_{k = m}^{r} ( \begin{matrix}r \\ k\end{matrix} ) {( 1 - C ( f ) )}^{k} {C ( f )}^{( r - k )}$$
where
r is the number of sample sources;
k is a particular sample source; and
C(f) is a cumulative distribution function for consistency scores f(g;m).

18. The method of claim 17 wherein the cumulative distribution function C(f) corresponds to an assumed normal distribution of the consistency scores f(g;m).

19. The method of claim 17 wherein the cumulative distribution function C(f) is an observed cumulative distribution function for consistency scores f(g;m).

20. Computer instructions that implement the method of claim 12 encoded in a computer readable medium.

21. A system that determines, from gene-expression data, a degree to which one or more genes are differentially expressed with respect to an event, the system comprising:
a receiving-and-storing component that receives gene-expression-level data obtained from a number of sample sources, the gene-expression-level data including, for each sample source, gene-expression levels prior to and following the event;
a difference-metric-computing component that, for each sample source, computes a difference-metric for a number of genes; and
a scoring component that employs difference-metrics produced by the difference-metric computing component to compute a rank-based consistency score for one or more genes, each consistency score reflective of the degree to which a gene is differentially expressed with respect to the event, and that computes a significance level for each consistency score.

22. The system of claim 21 wherein the difference-metric-computing component computes a difference-metric for a gene i, Dₖ(i) by:
$${D}_{k} ( i ) = \frac{1}{\vert {C}_{1} \vert} {\sum }_{j ε {C}_{k , 1}} {E}_{i , j}^{k} - \frac{1}{\vert {C}_{2} \vert} {\sum }_{j ε {C}_{k , 2}} {E}_{i , j}^{k}$$
where
Dₖ(i) is the difference metric for gene i computed for sample source k;

|C₁| is a number of gene-expression-level values in class 1;

|C₁| is a number of gene-expression-level values in class 2;
E_{i,j}^{k} is a log of the gene-expression-level value determined for gene i in sample j;
C_{k,1} is a class 1 partition of sample-source partition k; and
C_{k,2} is a class 2 partition of sample-source partition k.

23. The system of claim 21 wherein the scoring component employs the computed difference-metrics to compute a consistency score for one or more genes by:
sorting r vectors containing the computed difference-metrics for each sample source by the values of the difference-metrics in descending order to produce r rank vectors;
for each of the one or more genes,
computing a rank-consistency score s(g;m) as the m^{th} smallest rank for gene g in the r rank vectors.

24. The system of claim 23 wherein computing a significance level for each consistency score s(g;m) further includes computing p-Val(s,m) by:
$$p - Val ( s , m ) = {\sum }_{k = m}^{r} ( \begin{matrix}r \\ k\end{matrix} ) {{s}^{k} ( 1 - s )}^{( r - k )}$$
where
r is a number of sample sources; and
k is a particular sample source.

25. The system of claim 21 wherein the scoring component employs the computed difference-metrics to compute a consistency score for one or more genes by:
pooling r vectors containing the computed difference-metrics for each sample source and sorting the pooled difference-metrics to produce a pooled vector;
for each of the one or more genes,
computing a fold-consistency score f(g;m) as the m^{th} largest difference-metric for gene g in the pooled vector.

26. The system of claim 25 wherein computing a significance level for each consistency score f(g;m) further includes computing p-Val(s,m) by:
$$p - Val ( f ; m ) = {\sum }_{k = m}^{r} ( \begin{matrix}r \\ k\end{matrix} ) {( 1 - C ( f ) )}^{k} {C ( f )}^{( r - k )}$$
where
r is a number of sample sources;
k is a particular sample source; and
C(f) is a cumulative distribution function for consistency scores f(g;m).

27. The system of claim 26 wherein the cumulative distribution function C(f) corresponds to an assumed normal distribution of the consistency scores f(g;m).

28. The system of claim 27 wherein the cumulative distribution function C(f) is an observed cumulative distribution function for consistency scores f(g;m).

29. The system of claim 21 wherein the receiving-and-storing component, the difference-metric-computing component, and the scoring component are each implemented in one of:
hardware logic circuits;
firmware stored in a computer readable medium; and
software.
